(19) United States
(12) Patent Application Publication
(10) Pub. No.: US 2011/0182948 A1
(43) Pub. Date: Jul. 28, 2011

(54) METHOD FOR TREATING DISEASE CHARACTERIZED BY PLAQUE

(76) Inventors: Beka Solomon, Herzliyza (IL); Oded Grinstein, Tel Aviv (IL); Nir Friedman, Nes Ziona (IL); Michal Arbel, Gedena (IL); Haim Tsubery, Elad (IL); Richar Fisher, Cambridge, MA (US)

(21) Appl. No.: 12/993,393

(22) PCT Filed: May 22, 2009
(86) PCT No.: PCT/US09/45050

§ 371 Date: Apr. 13, 2011

Related U.S. Application Data
(60) Provisional application No. 61/055,311, filed on May 22, 2008.

(51) Int. Cl. A61K 9/00; A61K 38/02; A61K 35/76; A61P 25/00; A61P 25/28; A61P 25/16; B82Y 5/00

(52) U.S. Cl. 424/400; 514/1.1; 424/93.6; 514/17.7; 514/17.8; 977/762; 977/915

(57) ABSTRACT

The present invention relates to the use of a filamentous agent other than a filamentous bacteriophage to disaggregate aggregated proteins in plaque or to treat a patient suffering from or susceptible to a disease characterized by the presence of plaque.

### BACKGROUND OF THE INVENTION

1. Field of the Invention

The invention relates to methods for treating diseases and conditions characterized by plaque. In particular, the invention relates to the treatment of neurodegenerative diseases, such as Alzheimer's disease, Parkinson's disease, and Huntington's disease.

2. Description of the Related Art

Many progressive neurodegenerative diseases are morphologically characterized by the intracellular and/or extracellular presence of aggregated proteins, known as plaques, in the brain. The identity of the proteins present in plaques differs depending upon the disease. However, it is generally believed that disaggregation of the plaques and prevention of additional plaque formation is important in the treatment of the disease.

Alzheimer's disease (AD) is a progressive disease resulting in senile dementia. Broadly speaking, the disease falls into two categories: late onset, which occurs in old age (typically above 65 years) and early onset, which develops well before the senile period, e.g., between 35 and 60 years. In both types of the disease, the pathology is similar, but the abnormalities tend to be more severe and widespread in cases beginning at an earlier age. The disease is characterized by two types of lesions in the brain, senile plaques and neurofibrillary tangles. Senile plaques are areas of disorganized neutrophils up to 150 mm across with extracellular amyloid deposits at the center, visible by microscopic analysis of sections of brain tissue. Neurofibrillary tangles are intracellular deposits of tau protein consisting of two filaments twisted about each other in pairs.

The principal constituent of the senile plaques is a peptide termed amyloid beta (Aβ) or beta-amyloid peptide (βAP or βA). The amyloid beta peptide is an internal fragment of 39-43 amino acids of a precursor protein termed amyloid precursor protein (APP). Several mutations within the APP protein have been correlated with the presence of Alzheimer's disease (Goate et al, Nature 349:704 (1991)), valine717 to isoleucine; Chartier Harlan et al, Nature 353:844 (1991), valine717 to glycine; Murrell et al, Science 254:97 (1991), valine717 to phenylalanine; Mullan et al, Nature Genet. 1:345 (1992), a double mutation, changing lysine595-methionine596 to asparagine595-leucine596).

Such mutations are thought to cause Alzheimer's disease by increased or altered processing of APP to beta-amyloid, particularly processing of APP to increased amounts of the long form of beta-amyloid (i.e., Aβ1-42 and Aβ1-43). Mutations in other genes, such as the presenilin genes, PS1 and PS2, are thought indirectly to affect processing of APP to generate increased amounts of long form beta-amyloid (see Hardy, TINS, 20:154 (1997)). These observations indicate that beta-amyloid, and particularly its long form, is a causative element in Alzheimer's disease.

Parkinson's disease (PD) is a progressive neurodegenerative disease whose primary clinical features include motor abnormalities, such as resting tremor, bradykinesia and rigidity (Fahn, S., & Sulzer, D. Neurodegeneration and neuroprotection in Parkinson disease NeuroRx:, 1:139-154 (2004)). PD is characterized by the loss of dopaminergic neurons in the substantia nigra pars compacta and the presence of inclusion bodies, called Lewy bodies and Lewy neurites, in the surviving neurons of the same region (Forno, L. S. Neuropathology of Parkinson's disease. Journal of Neuropathology and Experimental Neurology, 55:259-272 (1996)). Although it is generally accepted that the loss of midbrain dopaminergic neurons is largely responsible for the major motor symptoms, this is not the only region showing pathologic changes in PD patients. Lewy pathology and cell loss first appear in lower brain stem nuclei, progressively ascend to the midbrain and finally to cortical areas in a highly predictable manner (Braak et al., 2004). Progression of the Lewy pathology to the various regions outside the midbrain may account for the abundance of the secondary symptoms commonly observed in PD patients, such as depression, dementia, and various autonomic and sensory dysfunctions.

Although the cause of PD remains elusive, there is a large body of evidence suggesting that misfolding and abnormal aggregation of α-synuclein is an important component of the disease pathogenesis. Genetic linkage analyses have identified three missense mutations in the inherited forms of parkinsonism (Kruger, R. et al. Nature Generics, 18:106-108 (1998); Polymeropoulos, M. H., et al, Science, 276:2045-2047 (1997); Zarranz, J J. et al., Annals of Neurology, 55:164-173 (2004)), and all the mutant variants have been shown to accelerate either oligomerization or fibrillation (Conway et al. 2000; Greenbaum, E. A. et al., Journal of Biological Chemistry, 280: 7800-7807 (2005)). Accumulation of wild type α-synuclein is sufficient to cause the disease.

Fibrillar aggregates of α-synuclein seem to be the main component of Lewy bodies and Lewy neurites, and these are now considered the most reliable PD marker for postmortem diagnosis (Spillantini, M. et al., PNAS (USA), 95:6469-6473 (1998)). Because α-synuclein is a cytosolic protein it has been assumed that the pathogenic changes and effects induced by the protein occur in the cytoplasm and are limited to the single cell. However, recent studies of extracellular α-synuclein suggest that the scope of pathogenic action goes beyond the cytoplasm of its origin (Lee, S. J., J. Mol. Neurosci., 34:17-22, 2008).

The presence of α-synuclein and its aggregated forms in extracellular fluid was recently demonstrated both in vivo and in vitro. Extracellular α-synuclein appears to be delivered by unconventional exocytosis of intravesicular α-synuclein, although the exact mechanism has'not been characterized. Intravesicular α-synuclein is prone to aggregation and is the potential source of extracellular aggregates.

The role of secreted α-synuclein in the extracellular space can be inferred from studies using tissue culture systems. Several studies reported cytotoxic effects of extracellular α-synuclein and its internal hydrophobic fragment (nonamyloid component or NAC) when the proteins were added to the culture medium (Albani et al. 2004; Bodles et al. 2000; Du et al. 2003; El-Agnaf et al. EBS Letters, 440:71-75 (1998); Forloni et al. Annals of Neurology, 47:632-640 (2000); Lee et al. Biochemistry, 43:3704-3715 (2004); Seo et al. MSEB Journal, 16:1826-1828 (2002); Sung et al. Journal of Biological Chemistry, 276:27441-27448 (2001)). Some studies have demonstrated the toxic effect of fibrillar aggregates (Bodies et al. 2000; El-Agnaf et al. supra), while other studies identified protofibrillar or oligomeric aggregates as the toxic culprit (Du et al. 2003).

Alpha synuclein can readily incorporate into membranes and can be found in synaptic vesicles and on the cell membrane. There are not many well-structured models for the mechanisms of toxicity. Recent studies illustrate a possible role for an α-synuclein pore-like protofibrils in the pathogenesis of Parkinson's disease (Tsigelny et al., FEBS Journal 274, 1862-1877 (2007); Lee et al., J. Biol. Chem. 277, 671-678 (2002); Volles and Lansbury, Biochemistry 41, 4595-4602 (2002)). One model proposes that oligomeric α-synuclein can form annular structures with a central pore (Volles and Lansbury Biochemistry, 40:7812-7819 (2003)). These aggregates can bind to membranes (Volles et al. 2001) and their membrane permeabilizing action has been demonstrated in synthetic model membranes, such as phospholipid liposomes (Volles et al. 2001) and planar bilayer membranes (Kayed et al. 2004). Insertion of these aggregates into the cell membrane would have a catastrophic effect on cell viability due to the free exchange of ions and small metabolites between the cytoplasm and the extracellular space. Although this toxic pore model explains the cytotoxicity of at least some oligomeric aggregates, the pores and the pore activity have yet to be demonstrated in biological systems. Another potential mechanism of neurotoxicity of extracellular α-synuclein, and especially its aggregate forms, may involve neuroinflammatory responses (Zhang et al., FASEB Journal, 19:533-542 (2005); Klegeris et al., FASEB Journal, 20:2000-2008 (2006)).

Huntington's disease (HD) is a genetic neurological disorder characterized by abnormal body movements called chorea and a lack of coordination. It also affects a number of mental abilities and some aspects of behavior. In more advanced stages it can cause complications that significantly reduce life expectancy. There is currently no proven cure for HD.

Huntington's disease (HD) is caused by the expansion of a CAG repeat in the huntington gene, which results in the expression of a mutant form of the protein, huntingtin (htt), with expanded Glu repeats that is toxic to neurons. Several mechanisms have been identified in mediating this toxicity, such as protein aggregation, mitochondrial dysfunction, oxidative stress, transcriptional dysregulation, aberrant apoptosis, altered proteosomal function and excitotoxicity.

Other peptides or proteins with evidence of self aggregation are also known, such as, but not limited to, amylin (Young et al, 1994); bombesin, cerulein, cholecystokinin octapeptide, eledoisin, gastrin-related pentapeptide, gastrin tetrapeptide, somatostatin (reduced), substance P, luteinizing hormone releasing hormone, somatostatin N-Tyr (Banks and Kastin, 1992). Other diseases hallmarked by the presence of protein aggregates and related to neurodegeneration include prion diseases, amyotrophic lateral sclerosis, spinocerebellar ataxia, dentatorubral-pallidoluysian atrophy, spinal and bulbar muscular atrophy, hereditary cerebral amyloid angiopathy, familial amyloidosis, Frontotemporal lobe dementia, British/Danish dementia, and familial encephalopathy.

It has previously been demonstrated that filamentous bacteriophage, in particular M13, can disaggregate β-amyloid plaque (WO2006083795 and WO2008011503), α-synuclein aggregates (WO2008011503), and huntingtin plaque (unpublished data). Despite the advances made to date in developing therapies to treat some of these diseases, there is still a great need for additional therapies.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention provides methods for disaggregating aggregated proteins (“plaque”). The methods utilize a filamentous agent to treat a patient suffering from or susceptible to a disease characterized by the presence of plaque. The method involves administering to the patient a filamentous agent other than a filamentous bacteriophage that (a) has a helical structure comprising repeated protein or peptide subunits; (b) has a length of 100 to 5,000 nm; (c) has a width of 2 to 20 nm; and (d) has a length-to-width ratio of 10 or higher.

Without being bound by theory, the present inventors believe that the agents utilized in the methods according to the present invention are capable of disaggregating plaque due to a physical and conformational similarity to the component(s) of the plaque and their ability to associate with existing plaque. It is believed that the interaction is not specific to any particular plaque protein, but rather to the general structure that is common to plaques. Accordingly, the methods disclosed herein are useful to disaggregate almost any type of plaque and to treat diseases characterized by almost any type of plaque.

### BRIEF DESCRIPTION OF THE DRAWINGS

FIG. 1 is a graph of ThT fluorescence at approximately 485 nm of Aβ aggregates after incubation in the presence of various dilutions (titers) of M13 phage. Controls for M13 phage in the absence of Aβ aggregates are also shown.

FIG. 2 is a graph of ThT fluorescence from FIG. 1 after subtracting the ThT fluorescence of the M13 phage in the controls.

FIG. 3 is a graph of the percentage disaggregation of Aβ aggregates based on ThT fluorescence after incubation in the presence of various titers of M13 phage.

FIG. 4 is a graph of ThT fluorescence at approximately 485 nm of Aβ aggregates after incubation in the presence of various dilutions (concentrations) of E. coil type 1 pili. The controls are ThT only, Aβ only and pili only.

FIG. 5 is a graph of the percent Aβ disaggregation (based on ThT fluorescence) after incubation in the presence of various dilutions (concentrations) of pili.

FIG. 6 is a graph of absorbance (OD) at 492 nm of Aβ aggregates after incubation in the presence or absence (negative control) of various titers of M13 phage in the ELISA Trap assay.

FIG. 7 is a graph of absorbance (OD) at 492 nm of Aβ aggregates after incubation in the presence or absence (negative control) of various concentrations of pili in the ELISA Trap assay. Controls for pili in the absence of Aβ aggregates are also shown.

FIG. 8 is a graph of absorbance (OD) at 492 nm of Aβ aggregates after incubation in the presence or absence (negative control) of various titers of TMV in the ELISA Trap assay. Controls for TMV in the absence of Aβ aggregates are also shown.

### DETAILED DESCRIPTION OF THE INVENTION

### Definitions

For purposes of this specification and the accompanying claims, the following definitions apply.

The terms “patient”, “subject” and “recipient” are used interchangeably. They include humans and other mammals which are the object of therapeutic treatment.

The term “treating” with respect to a disease characterized by the presence of plaque is intended to mean substantially inhibiting, slowing or reversing the progression of the disease, such as reducing or inhibiting the formation of plaque, or disaggregating pre-formed plaque, and optionally reducing or inhibiting the formation of plaque; substantially ameliorating one or more clinical symptoms of the disease; or substantially preventing the appearance of clinical symptoms of the disease.

The term “co-administer” is intended to mean administration by means of a single dosage form or by means of multiple dosage forms administered simultaneously, sequentially or separately. Preferably, co-administration causes the effects of each administration to be exerted on the cells being treated at an overlapping period of time, more preferably simultaneously.

The term “pro-inflammatory cytokine” refers to any proinflammatory cytokine involved in brain inflammation associated with a disease that is characterized by the presence of plaque, including but not limited to IL-6, IL-1, IL-17 and TNFα.

The term “mammalian cell internalization signal” refers to any cell adhesion sequence which facilitates internalization as a result of cell adhesion/attachment to the cell. Numerous mammalian cell adhesion sequences are known and include the Arg-Gly-Asp (RGD) cell adhesion sequence, the Tat peptide from HIV and peptides comprising the sequence of Arg-Glu-Asp (RED), Arg-Lys-Lys (RKK), Leu-Asp-Val (LDV; Humphries, 1992), Leu-Leu-Gly (LLG; Koivunen et al., 2001), Asp-Gly-Glu-Ala (DGEA; SEQ ID NO:1), Ile-Arg-Val-Val-Met (IRVVM; SEQ ID NO:2; Kosfeld et al., 1993), Pro-His-Ser-Arg-Asp (PHSRN; SEQ ID NO:3) and RFYVVMWK (SEQ ID NO:4; Kosfeld et al., 1993). Many cell adhesion sequences (also known as cell attachment motifs) are known in cell adhesive molecules such as laminin, fibronectin, vitronectin, fibrinogen, thrombospondin, etc.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to a patient.

The phrases “physiologically acceptable carrier” and “pharmaceutically acceptable carrier,” which may be interchangeably used, refer to a carrier or a diluent that does not cause significant irritation to an organism and does not abrogate the biological activity and properties of the administered compound.

The term “excipient” refers to an inert substance added to a pharmaceutical composition to further facilitate administration of an active ingredient. Non-limiting examples of excipients include calcium carbonate, calcium phosphate, various sugars and types of starch, cellulose derivatives, gelatin, vegetable oils and polyethylene glycols.

### The Filamentous Agent

The filamentous agent used in the methods of the present invention is an agent other than a filamentous bacteriophage that (i) has a helical structure comprising repeated protein or peptide subunits; (ii) has a length of 100 to 5,000 nm; (iii) has a width of 2 to 20 nm; (iv) has a length-to-width ratio of 10 or higher.

ThT fluorescence methods are well-known in the art and are utilized to identify extended β-sheet formations, such as those found in dense core β-amyloid plaque.

The filamentous agents used in this invention must be in a disaggregated state in order to effectively disaggregate plaque. This is typically achieved by providing a composition of such agents below the concentration at which those agents begin to aggregate. While the actual concentration will vary depending upon the nature of the agent, it is well within the skill of the art to determine whether or not the agent is aggregated through turbidity measurements and other standard assays.

The filamentous agent utilized in the methods of the present invention may be of either biological or synthetic origin. For example, the filamentous agent comprising one or more proteins may be a virus, such as tobacco mosaic virus. Alternatively, the filamentous agent may be a portion of an organism having the requisite structural parameters and properties. Examples of this type of filamentous agent include bacterial pili/fimbriae (e.g., E. coli type 1 pili, P pili, K-99 pili, S pili and 987P fimbriae), and bacteriophage T4 tail. Some of the structural properties of M13, E. coli type 1 pili, TMV and bacteriophage T4 tail are shown in Table 1.

TABLE 1

| | | Contains | | | | Nanotubular | α-helical |
|---|---|---|---|---|---|---|---|
| | helical | repeating | Length | Width | L:W | structure | structural |
| Agents | cylinder | subunits | (nm) | (nm) | ratio | portion | component |
| M13 | yes | yes | 900-960 | 4-6 | >150 | yes | yes |
| E. coli | yes | yes | 500-2000 | 7 | >70 | yes | yes (right- |
| type 1 | | | | | | | handed) |
| pili | | | | | | | |
| TMV | yes | yes | 300 | 12-18 | 25 | yes | yes |
| **T4 | yes | yes | 100 | 10 | 10 | yes | complex |
| tail tube; | | | | | | | |
| (Tail has | | | | | | | |
| variable L) | | | | | | | |

**T4 tail is contractile so length of whole tail varies; the inner tail tube dimensions are given here.

In certain embodiments, at least a portion of the filamentous agent will have a nanotubular structure. In other embodiments, at least a portion of the filamentous agent interacts with Tht to induce Tht fluorescence. In still other embodiments, at least a portion of the filamentous agent will exhibit an α-helical structural component, subunit, or portion with an α-helical fold.

In certain embodiments, the filamentous agent may comprise a mammalian cell internalization signal. Such a signal may be genetically engineered into those filamentous agents that are of biological origin by techniques well-known in the art. For example, an RGD mammalian cell internalization signal can be inserted into one of the structural proteins that make up TMV, thus allowing the TMV to cross the mammalian cell membrane and disaggregate intracellular plaque.

### Methods of Treatment

In one embodiment, the invention provides a method of disaggregating existing plaque comprising the step of contacting the plaque with a filamentous agent other than a filamentous bacteriophage that (i) has a helical structure comprising repeated protein or peptide subunits; (ii) has a length of 100 to 5,000 nm; (iii) has a width of 2 to 20 nm; and (iv) has a length-to-width ratio of 10 or higher, wherein the filamentous agent is disaggregated prior to contacting the plaque. In one aspect of this embodiment, the existing plaque comprises one or more proteins selected from huntingtin, α-synuclein, TAU protein, β-amyloid, prion protein (PrP), SOD1, ataxin-1, ataxin-3, ataxin-7, a calcium channel protein, atrophin-1, cytastatin-3 or a fragment thereof, transthyretin, lysozyme, an androgen receptor, a briPP protein fragment, neuroserpin, amylin, bombesin, cerulein, cholecystokinin octapeptide, eledoisin, gastrin-related pentapeptide, gastrin tetrapeptide, somatostatin (reduced), substance P, luteinizing hormone releasing hormone, somatostatin N-Tyr. The reference to each of the proteins recited above also includes a reference to naturally occurring mutant forms of those proteins. In many instances, it is those naturally occurring mutant forms that aggregate and form plaques.

In a more specific aspect, the existing plaque comprises one or more proteins selected from huntingtin, α-synuclein, TAU protein, β-amyloid, prion protein (PrP), and SOD1. In an even more specific aspect, the existing plaque comprises one protein selected from huntingtin, α-synuclein, TAU protein, and β-amyloid. In a further specific aspect, the existing plaque comprises β-amyloid.

In another embodiment, the invention provides a method of treating a patient suffering from or susceptible to a disease characterized by the presence of plaque comprising the step of administering to the patient a filamentous agent other than a filamentous bacteriophage that (i) has a helical structure comprising repeated protein or peptide subunits; (ii) has a length of 100 to 5,000 nm; (ii) has a width of 2 to 20 nm; and (iii) has a length-to-width ratio of 10 or higher.

In one aspect of this embodiment, the method is used to treat a patent suffering from or susceptible to a neurodegenerative disease. In a more specific aspect the neurodegenerative disease is selected from Alzheimer's Disease, Parkinson's Disease, Huntington's Disease, a prion disease, amyotrophic lateral sclerosis, spinocerebellar ataxia, dentatorubral-pallidoluysian atrophy, spinal and bulbar muscular atrophy, hereditary cerebral amyloid angiopathy, familial amyloidosis, Frontotemporal lobe dementia, British/Danish dementia, and familial encephalopathy. In an even more specific aspect, the neurodegenerative disease is selected from Alzheimer's Disease, Parkinson's Disease, Huntington's Disease, and amyotrophic lateral sclerosis. In a further specific aspect of this embodiment, the disease is Alzheimer's Disease.

In certain embodiments, the filamentous agent prevents aggregation of proteins into plaque. This feature is in addition to the ability of the filamentous agent to disaggregate plaque and is a feature that is useful in all of the diseases and conditions set forth above.

In one embodiment, the filamentous agent is administered to the patient by intranasal administration. It is known that intranasal administration of filamentous bacteriophage results in the bacteriophage crossing the blood-brain barrier (WO2006083795). These phage are then eliminated from the brain and body via urine and feces without adverse effects on peripheral organs. Without being bound by theory, the inventors believe that some or all of the filamentous agents utilized in this invention may also be able to cross the blood-brain barrier upon intranasal administration.

In an alternate embodiment, the filamentous agent is administered to the patient by intracranial administration.

In certain embodiments, the therapeutic methods of this invention comprise the additional step of co-administering to the patient in need thereof a second therapeutic agent useful to treat the disease or condition from which the patient is suffering or to which the patient is susceptible. Such second therapeutic agents include antibodies to the protein(s) that make up the plaques, antibodies to pro-inflammatory cytokines, dimebolin hydrochloride, donepezil, memantine, L-dopa, carbidopa, a dopamine agonist, an anticholinergic agent, a COMT inhibitor, an anti-inflammatory agent, or combinations thereof.

Methods delineated herein also include those wherein the patient is identified as in need of a particular stated treatment. Identifying a patient in need of such treatment can be in the judgment of a patient or a health care professional and can be subjective (e.g., opinion) or objective (e.g., measurable by a test or diagnostic method).

### Pharmaceutical Compositions

In a related embodiment, the invention provides a pharmaceutical composition (e.g., pyrogen-free) comprising: (a) a filamentous agent other than a filamentous bacteriophage, the filamentous agent comprising the following properties: (i) a helical structure comprising repeated protein or peptide subunits; (ii) a length of 100 to 5,000 nm; (iii) a width of 2 to 20 nm; and (iv) a length-to-width ratio of 10 or higher; and (b) a pharmaceutically acceptable carrier, wherein the filamentous agent is not aggregated in the composition.

The filamentous agent may have any of the properties set forth above and may be selected from any of the agents set forth above. In one specific embodiment, the filamentous agent is selected from E. coil type 1 pili and tobacco mosaic virus.

In one embodiment, the composition is formulated for intranasal administration. In another embodiment, the composition is formulated for intracranial administration.

Techniques for formulation and administration of drugs may be found in “Remington's Pharmaceutical Sciences,” Mack Publishing Co., Easton, Pa., latest edition, which is incorporated herein by reference and are well known in the art.

Pharmaceutical compositions for use in accordance with the present invention thus may be formulated in a conventional manner using one or more physiologically acceptable carriers comprising excipients and auxiliaries, which facilitate processing of the active ingredients into preparations which can be used pharmaceutically.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. A nasal spray, which does not require a pressurized pack or nebulizer as in an inhalation spray, can alternatively used for intranasal administration. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch. In certain embodiments, the pharmaceutically acceptable carrier is a sterilized saline solution.

Pharmaceutical compositions suitable for use in the context of the method of the present invention include compositions wherein the active ingredient(s) is contained in an amount effective to achieve the intended purpose. More specifically, an effective amount means an amount of active ingredient(s) effective to treat Parkinson's disease.

Determination of a therapeutically effective amount is well within the capability of those skilled in the art, especially in light of the detailed disclosure provided herein.

Dosage amount and interval may be adjusted, individually to provide brain levels of the filamentous agent which are sufficient to treat the target disease or condition (minimal effective concentration, MEC). The MEC will vary for each preparation, based upon the identity of the filamentous agent and the nature of the disease, but can be estimated from in vitro data. Dosages necessary to achieve the MEC will depend on individual characteristics of the patient being treated.

Dosage intervals can also be determined using the MEC value. Preparations should be administered using a regimen, which maintains brain levels above the MEC for 10-90% of the time, preferably between 30-90% of the time and most preferably between 50-90% of the time during the course of treatment.

Depending on the severity and responsiveness of the disease to be treated in the patient, dosing can be of a single or a plurality of administrations, with the course of treatment lasting from several days to several weeks or until diminution of the disease state is achieved.

The amount of a composition to be administered will, of course, be dependent on the subject being treated, the severity of the affliction, the judgment of the prescribing physician, etc.

Compositions used in the method of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Having now generally described the invention, the same will be more readily understood through reference to the following Examples which are provided by way of illustration and are not intended to be limiting of the present invention. The experimental results in the examples below demonstrate the effect of various filamentous agents on disaggregation of amyloid-beta aggregates involved in the pathogenesis of AD.

### EXAMPLE 1

### ThT Assay for Quantification of Filamentous Agent Disaggregation Activity

Thioflavin (ThT) assay is a common tool to quantify the formation of amyloid fibril of Aβ (Levine, 1999). The assay is based on the fluorescence shift of thioflavin upon interaction with β-sheet structure. Aggregated Aβ forms a β-sheet structure that is lacking in the monomer state of the peptide. Interaction with ThT molecules induces a specific fluorescence of ThT at −485nm. This assay is used to follow aggregation and disaggregation of Aβ. If a substance disaggregates Aβ fibrils, then the ThT fluorescence is reduced relative to intact Aβ fibrils.

### Filamentous Bacteriophage

Aβ1-40 preparation: 1 mg Aβ1-40 (Bachem, H-1194) is dissolved in 1 ml acetonitrile: water:trifluoroacetic acid (80:20:0.1%/v:v:v) and aliquoted to sterile 1 ml tubes (100 μg peptide/tube). Samples are frozen in liquid nitrogen and lyophilized. Dry samples are kept sealed with parafilm at −20° C.

Aβ1-40 aggregation: Aβ40 (100 ug, 50 uM) is dissolved in DMSO (30 ul) and diluted in ultra pure water (435 μl) for 14 days at 37° C. An alternate method allows Aβ40 (10 μl, 0.5 ml tube) to aggregate for 3-4 weeks.

Disaggregation Assay Reaction: Aggregated Aβ40 (5 μl/well of 50 μM solution) is aliquoted to a microtiter plate containing triplicates of M13 phage (45 μl/well) at 5 fold dilutions (5×10¹³, 1×10¹³, 2×10¹², 4×10¹¹ and 8×10¹⁰ phage/ml). PBS, Aβ40 alone and WT M13 alone were used as a control. The microtiter plate is sealed with aluminum foil to prevent evaporation and is incubated for 48 hr at 37° C.

Detection of β-Amyloid: The microtiter plate is spun down to minimize volume differences between wells. ThT solution (150 μl/well, 5 μM in 50 mM Glycine buffer, pH8.5) is added to each well and the plate is read immediately (430 nm/492 nm).

### Results

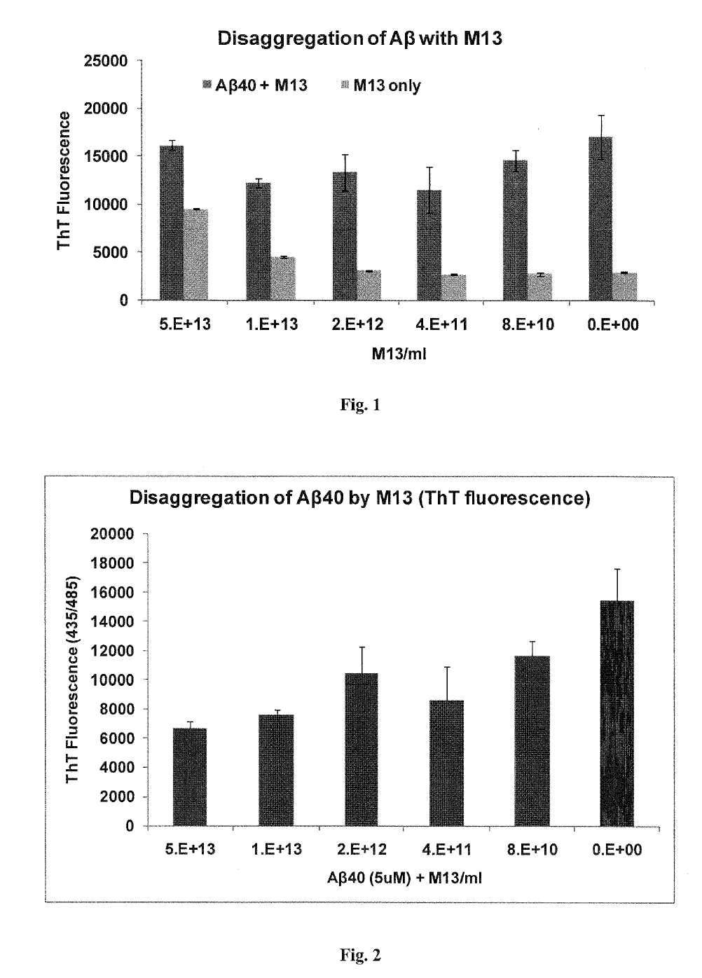

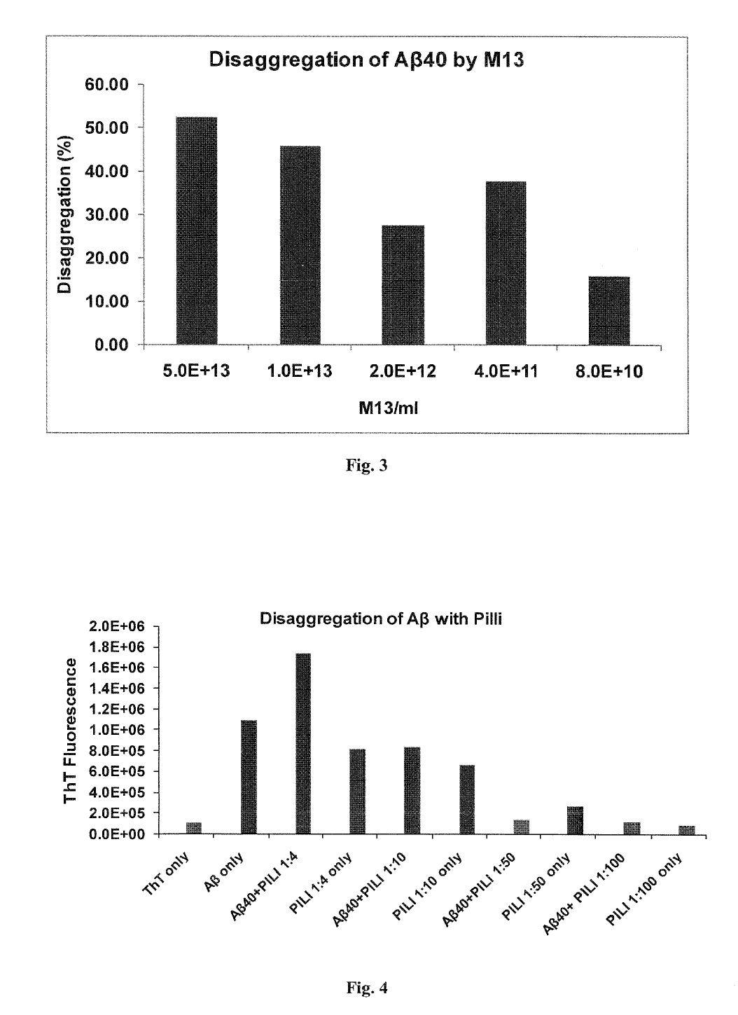

The results of the Aβ disaggregation assay (by measuring ThT induced fluorescence at approximately 485 nm) with various dilutions of M13 phage are shown in FIG. 1. FIG. 2 shows the results of Aβ disaggregation based on ThT fluorescence after subtracting out the ThT fluorescence of M13 phage controls. As can be seen from FIG. 2, increasing phage titers results in decreasing ThT fluorescence, which is indicative of Aβ disaggregation. FIG. 3 shows the percent disaggregation of Aβ by M13 phage at various titers of phage.

### Bacterial Pili

### Preparation of Bacterial Pili

Bacterial Growth: The bacteria are grown as described elsewhere (Eshdat et al., 1981) with the following modifications: A starter culture of E. coli 346 strain is grown in LB medium with shaking overnight at 37° C. Then a 1:100 dilution is made by adding to fresh LB medium (1 L of medium with bacteria into a 5 L flask in order to increase the surface area and the oxygen supply). Finally, the culture was grown without shaking for 48 hr at 37° C. In order to maximize the type 1 pili yield, several passages are made (3-4 times), using fresh LB medium for each passage.

After a few passages, the bacteria are harvested by centrifugation at 7000 rpm for 7 min. The bacterial pellet is re-suspended in 160 ml of 5 mM Tris/75 mM NaCl buffer (pH=8) and kept at 4° C.

Type 1 pili purification: Purification of type 1 pili is made using a mechanical agitation and precipitation in salts. This procedure is described elsewhere (Eshdat et al., 1981; Slonim et al., 1992) and is used in this work with several modifications.

The centrifuged and washed bacteria are disrupted to remove pili using a blender on ice for 1.5 min. with 1 min. intervals without blending on ice (in order to prevent overheating); the sample is blended a second time for an additional 1.5 min. The blended sample is centrifuged at 8,000 rpm for 10 min at 4° C. to pellet de-fimbriated cells. The supernatant is poured into clean tubes and centrifuged again at 15,000 rpm for 30 min at 4° C. in order to remove cell debris. The pili supernatant is filtered through a 0.45 μm spinning top filter. The filtered solution is poured into a 100 ml graduated cylinder with a stirrer and is gently stirred on ice. NaCl solution (5M) is then added dropwise using a Pasteur pipette to a final concentration of 300 mM. MgCl₂ (1M) is added dropwise using a Pasteur pipette to a final concentration of 100 mM. The sample is stirred at 4° C. for 2 hr, then poured into small tubes and centrifuged at 19,500 rpm for 1 hr at 4° C. The pellet comprised of the pili is suspended in 3 ml of 5 mM Tris buffer (pH=8) at 4° C. overnight. The next day the sample is spun at 20,000 rpm for 60 min at 4° C. Pili are in the supernatant. To remove pili remaining in the pellet, the steps of suspending the pellet and centrifuging are repeated. The 3 ml sample containing the type 1 pili is filtered through a 0.45 μm spinning top filter and concentrated to 0.7-1 ml using “Eppendorf concentrator 5301” to a final stock solution and stored at 4° C.

Protein quantification: BCA kit is used in order to quantify the concentration of the pili. The stock solution is adjusted to 3.5 mg/ml final protein concentration.

Aβ1-40 preparation: Aβ40 (Bachem, H-1194, 1 mg) is dissolved in DMSO (1 ml) and kept at −20° C.

Aβ1-40 aggregation: Aβ40 solution in DMSO is diluted in ultra pure water to a final peptide concentration of 50 μM. 10 μl aliquots in 0.5 ml sterile tubes are incubated for 14-21 days at 37° C.

Disaggregation Assay Reaction: Pili (10 μl/tube of 10-fold serial dilutions) is added to aggregated Aβ40 solutions-containing tubes (10 μl/tube), mixed gently and incubated overnight at 37° C.

Detection of β-Amyloid: ThT solution (500 μl, 5 μM in 50 mM Glycine buffer, pH8.5) is added to each reaction tube, mixed and transferred to quartz cuvettes. Fluorescence is measured and recorded immediately at 435 nm and 482 nm, excitation and emission wavelengths, respectively.

### Results

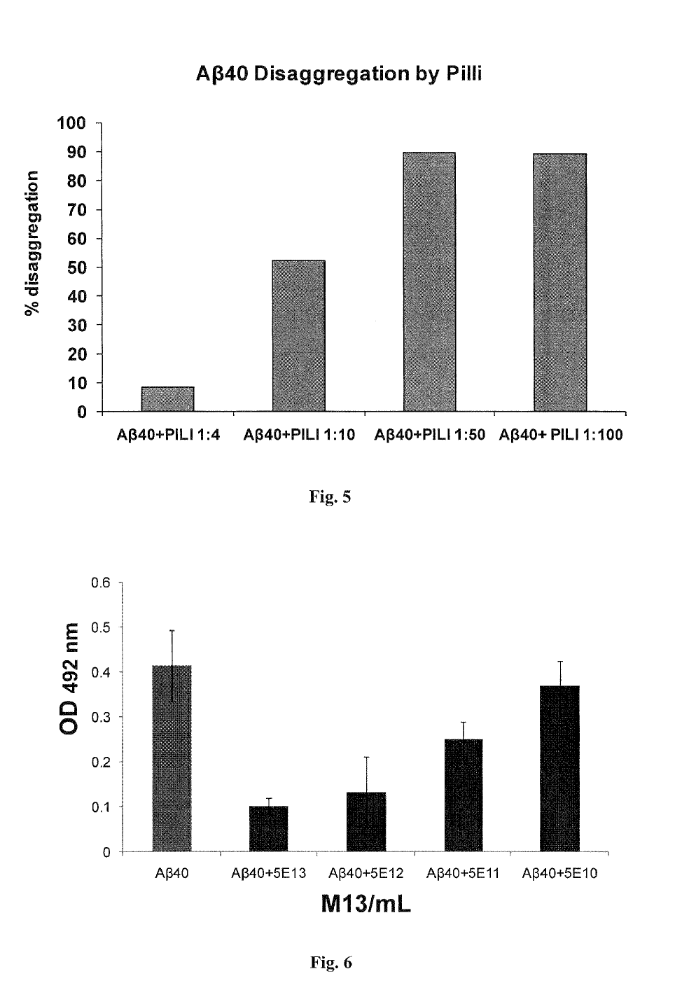

The results of the Aβ disaggregation assay (by measuring ThT induced fluorescence at approximately 485 nm) with various dilutions of bacterial pili are shown in FIG. 4. In FIG. 4, the lower concentrations of pili result in decreasing ThT fluorescence and an increase in the percent disaggregation of Aβ (FIG. 5).

### EXAMPLE 2

### ELISA Trap Protocol for Measuring Aggregated Aβ (β-Amyloid)

This ELISA Trap assay is also useful for detecting disaggregation. It measures uniquely polyvalent β-amyloid, and is based oh an assay published by LeVine (2004). By comparing β-amyloid aggregates remaining after incubation with a putative disaggregating agent, such as M13, TMV, and fimbriae, compared to aggregates remaining after incubation with a negative control, such as saline, it is possible to measure the extent of disaggregation. In other words, this is a useful assay for screening agents that promote amyloid disaggregation.

Aβ1-40 preparation: Aβ1-40 (Bachem, H-1194, 1 mg) is dissolved in 1 ml acetonitrile: water: trifluoroacetic acid (80:20:0.1% v:v:v) and aliquoted to sterile 1 ml tubes (100 μg peptide/tube). Samples are frozen in liquid nitrogen and lyophilized. Dry samples are kept sealed with parafilm at −20 C.

Aβ1-40 aggregation: Aβ40 (100 μg, 50 μM) is dissolved in DMSO (30 μl) and diluted in ultra pure water (435 μl) for 14 days at 37° C.

Disaggregation Assay Reactions:

M13 phage: Aggregated Aβ40 is aliquoted to Eppendorf (0.5 ml) tubes (15 μl each). M13 (15 μl, PBS solution of 1×10¹⁴, 1×10¹³, 1×10¹², 1×10¹¹/ml) are added in triplicates to the Aβ40 containing tubes. Reaction tubes are mixed gently, spin down for 10 sec and incubate at 37° C. for 48 hr.

Pili (Fimbria): Aggregated Aβ40 is aliquoted to Eppendorf (0.5 ml) tubes (15 μl each). Purified pili (35 μg/ml, 15 μl, PBS solution of serial 10 fold dilutions) are added in triplicates to the Aβ40 containing tubes. Reaction tubes are mixed gently, spun down for 10 sec and incubated at 37° C. for 48 hr.

Tobacco Mosaic Virus (TMV): Aggregated Aβ40 is aliquoted to Eppendorf (0.5 ml) tubes (15 μl each). TMV and (15 μl, PBS solution of serial 10-fold dilutions of 1×10¹⁴, 1×10¹³, 1×10¹², 1×10¹¹/ml in PBS and a gift from Prof. Abed Gera, Institute of Plant Protection, Volcani Center, ISRAEL) is added in triplicates to the Aβ40-containing tubes. Reaction tubes are mixed gently, spun down for 10 sec and incubated at 37° C. for 48 hr.

Preparation of assay plates: Microtiter plates (Maxisorb, NUNC) are coated (50 μl/well) with 6E10 antibody (anti-amyloid N-terminal MAb, Covance, 1 mg/ml, diluted 1:500) in 0.1M Carbonate buffer pH-9.6, for 2 hr at 37° C. The plates are washed with 0.1% Tween 20 in PBS. The plates are blocked with 3% BSA in PBS (200 μl/well) for 16 hr at 4° C. and then washed with 0.1% Tween-20 in PBS.

Addition of Disaggregation Assay Test Samples: Samples for testing (disaggregation assay reactions) diluted 1:6 in PBS (150 μl to each sample tube, 50 μl/well) are added in triplicates, incubated at 37° C. for 3 hr and washed with 0.1% Tween 20 in PBS.

Detection of β-Amyloid: Biotin-labeled 6E10 antibody (Covance, 1 mg/ml) is diluted 1:1500 in 1% BSA in PBS and it is added to the wells (50 μl/well), followed by incubation for 1 hr at 37° C. Wells are washed with 0.1% Tween-20 in PBS. Streptavidin-HRP (Sigma) diluted 1:1000 in 1% BSA in PBS is added (50 μl/well), and plates are incubated for 1 hr at 37° C. OPD (100 μl/well, 15 mg/7.5 ml of 0.05M Citric acid pH 5.5/3 μl H₂O₂) is added, and the reaction is stopped with 4N HCl (50 μl/well). Absorbance at 492 nm is measured by spectrophotometry.

### Results

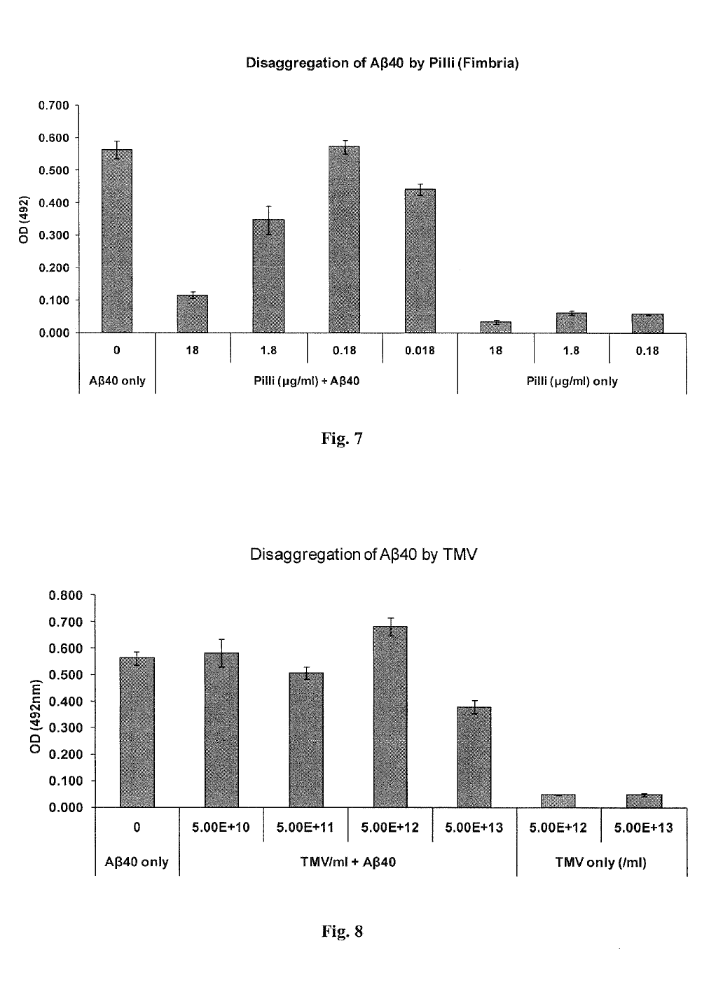

FIGS. 6, 7 and 8, respectively, show that like M13, filamentous agents according to the present invention such as E. coli type 1 pili and TMV, disaggregated Aβ aggregates at the higher concentrations/titers of the filamentous agent tested in this example.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Benhar et al., Phage display of single-chain antibodies. In: J. Colligen (Ed), Current Protocols in Immunology, Vol. 10.19B, John Wiley & Sons, Inc, USA (2001)
Berdichevsky et al., J. Immunol. Methods, 228:151-62 (1999)
Brent et al., Current Protocols in Molecular Biology, John Wiley & Sons Inc. (2003)
El-Agnaf, 0 M, Jakes, R, Curran, M D, Middleton, D, Ingenito, R., Bianchi, E, et al. Aggregates from mutant and wild-type alpha-synuclein proteins and NAC peptide Induce apoptotic cell death in human neuroblastoma cells by formation of beta-sheet and amyloid-like filaments. FEBS Letters, 440:71-75 (1998).
El-Agnaf, O. M. A. et al., FASEB J. 20, 419-425 (Mar. 1, 2006, 2006).
Eshdat, Y., Fredric J. Silverblatt and Nathan Sharon Dissociation and reassembly of E. coli type 1 pili. Journal of Bacteriology, 148:308-314 (1981)
Fahn, S., & Sulzer, D. Neurodegeneration and neuroprotection in Parkinson disease NeuroRx:, 1:139-154 (2004).
Forloni, G., Bertani, I., Calella, A. M., Thaler, F., & Invernizzi, R. Alpha-synuclein and Parkinson's disease: Selective neurodegenerative effect of alpha-synuclein fragment on dopaminergic neurons in vitro and in vivo, Annals of Neurology, 47:632-640 (2000).
Forno, L. S. Neuropathology of Parkinson's disease. Journal of Neuropathology and Experimental Neurology, 55:259-272 (1996).
Greenbaum, E. A. Graves, C. L., Mishlzen-Eberz, A. J., Lupoli, M. A., Lynch, D. R., Englander, S. W., et al. The E46K mutation In alpha-synuclein increases amyloid fibril formation. Journal of Biological Chemistry, 280: 7800-7807 (2005).
Humphries, Peptide recognition motifs involved in the binding of integrins to their ligands, Kidney International, 41:645-649 (1992)
Kay et al., Phage Display of Peptides and Proteins, A Laboratory Manual, Academic Press (1996)
Klegeris, A., Giasson, B. I., Zhang, H., Maguire, J., Pelech, S., & McGeer, P. L. Alpha-synuclein and its disease-causing mutants induce ICAM-1 and IL-6 in human astrocytes and astrocytoma cells. FASEB Journal, 20:2000-2008 (2006).
Koivunen et al., “Inhibition of β₂ integrin-mediated leukocyte cell adhesion by leucine-leucine-glycine motif-containing peptides, The Journal of Cell Biology, 153(5):905-915 (2001)
Kosfeld et al., “Identification of a new cell adhesion motif in two homologous peptides from the COOH-terminal cell binding domain of human thrombospondin” The Journal of Biological Chemistry, 268(12):8808-8814 (1993)
Kruger, R., Kuhn, W., Muller, T., Woitalla, D., Graeber, M. Kosel. S, et al. Ala30Pro mutation in the gene encoding alpha synuclein in Parkinson's disease. Nature Generics, 18:106-108 (1998).
Lee. E N, Cho, H. J, Lee, C. H., Lee, D, Chung, K. C, & Palk, S. R. Phthalocyanine tetrasulfonates affect the amyloid formation and cytotoxicity of alpha-synuclein. Biochemistry, 43:3704-3715 (2004).
Lee, H. J., C. Choi, S.-J. Lee, J. Biol. Chem. 277, 671-678 (2002).
Lee, S. J., Origins and effects of extracellular §-synuclein: Implications in Parkinson's Disease, J. Mol. Neurosci., 34:17-22 (2008).
LeVine, H. III, Methods in Enzymology, 309:274-284 (1999)
LeVine, H. III, Anal. Biochem., 335:81-90 (2004)
Masliah et al., Effects of α-synuclein immunization in a mouse model of Parkinson's disease, Neuron, 46:857-868 (2005)
Polymeropoulos, M. H., Lavedan, C., Leroy, E., Ide, S. E., Dehejia, A, Dutra, A., et al. Mutation In the alpha-synuclein gene identified in families with Parkinson disease, Science, 276:2045-2047 (1997).
Saggio et al., Gene, 152:35-39 (1995)
Sambrook et al., Molecular cloning: A laboratory manual, Cold Spring Harbor Laboratory, Cold Spring Harbor, N.Y. (1989)
Seo, J. ti. Rah, J. C., Choi, S. H., Shin, J. K., Min, K, Kim, H. S., et al. Alpha-synuclein regulates neuronal survival via Bcl-2 family expression and P13iAkt kinase pathway. MSEB Journal, 16:1826-1828 (2002).
Slonim, L. N., Jerome S. Pinkner, Carl-Ivar Branden and Scott J. Hultgren. Interactive surface in the PapD chaperone cleft is conserved in pilus chaperone superfamily and essential in subunit recognition and assembly. EMBO Journal, 11:4747-4756 (1992).
Spillantini, M. G., Crowther, R. A., Jakes, R., Eiasegawa, M., Goedert, M. alpha-synuclein in filamentous inclusions of Lewy bodies from Parkinson's disease and dementia with lewy bodies Proceedings of the National Academy of Sciences of the United States of America, 95:6469-6473 (1998).
Sung, J. Y., Kim, J. Palk, S. R., Park, J. H., Ahn, Y. S., & Chung, K. C. Induction of neuronal cell death by Rab5A-dependent endocytosis of alpha-synuclein. Journal of Biological Chemistry, 276:27441-27448 (2001).
Tsigelny et al., FEBS Journal 274, 1862-1877 (2007).
Uppala and Koivunen, Chem. High Throughput Screen, 3:373-392 (2000)
Volles, M. J., & Lansbury Jr., P. T. Zeroing In on the pathogenic form of alpha-synuclein and its mechanism of neurotoxicity Parkinson's disease. Biochemistry, 42:7871-7878 (2003).
Volles, M. J, Lee, S.-J., Rochet, J. C., Shtileman, M D., Ding, T. T., Kessler, J. C., et al. Vesicle permeabilization by protofibrillar alpha-synuclein: Implications for the pathogenesis and treatment of Parkinson's disease. Biochemistry, 40:7812-7819 (2003).
Volles, M. J., P. T. Lansbury, Biochemistry 41, 4595-4602 (2002).
Zarranz, J J., Alegre, J., Gomez-Esteban, J. C, Lezcano, E, Ros, R, Ampuero, I. et al. The new mutation, E46K, of alpha-synuclein causes Parkinson and Lewy body dementia Annals of Neurology, 55:164-173 (2004).
Zhang, W, Wang, T, Pei, Z., Miller, D. S, Wu, X., Block, M L., et al. Aggregated alpha-synuclein activates microglia: A process leading to disease progression in Parkinson's disease FASEB Journal, 19:533-542 (2005).

1. A method of disaggregating existing plaque in a patient, comprising administering to the patient in need thereof an effective amount of a filamentous agent, wherein the filamentous agent is other than a filamentous bacteriophage and has a helical structure comprising repeated protein or peptide subunits, with a length of 100 to 5000 nm, a width of 2 to 20 nm, and a length-to-width ratio of 10 or higher.

2. The method of claim 1, wherein the filamentous agent further has a nanotubular structural component.

3. The method of claim 1, wherein at least a portion of the filamentous agent has an α-helical fold.

4. The filamentous agent method of claim 1, wherein at least a portion of the filamentous agent interacts with Thioflavin (ThT) to induce ThT fluorescence.

5. The method of claim 1, wherein the filamentous agent is administered intranasally.

6. The method of claim 1, wherein the filamentous agent is administered intracranially.

7-8. (canceled)

9. The method of claim 1, wherein the filamentous agent is selected from the group consisting of tobacco mosaic virus, pili/fimbriae, and bacteriophage T4 tail.

10. The method of claim 9, wherein the filamentous agent is pili/fimbriae selected from the group consisting of Escherichia coli type 1 pili, P pili, K-99 pili, S pili, and 987P fimbriae.

11. The method of claim 1, wherein the plaque comprises one or more proteins selected from the group consisting of huntingtin, α-synuclein, TAU protein, β-amyloid, prion protein (PrP), and SOD1.

12. The method of claim 1, wherein the patient is suffering from a neurodegenerative disease or condition.

13. The method of claim 15, wherein the disease or condition is selected from the group consisting of Alzheimer's Disease, Parkinson's Disease, Huntington's Disease, and amyotrophic lateral sclerosis.

14. The method of claim 13, wherein the disease is Alzheimer's Disease.

15-31. (canceled)

32. The method of claim 12, wherein the neurodegenerative disease or condition is selected from Alzheimer's Disease, Parkinson's Disease, Huntington's Disease, a prion disease, amyotrophic lateral sclerosis, spinocerebellar ataxia, dentatorubral-pallidoluysian atrophy, spinal and bulbar muscular atrophy, hereditary cerebral amyloid angiopathy, familial amyloidosis, Frontotemporal lobe dementia, British/Danish dementia, and familial encephalopathy.

33. A pyrogen-free, pharmaceutical composition comprising:
(a) a filamentous agent other than a filamentous bacteriophage, the filamentous agent comprising the following properties:
(i) a helical structure comprising repeated protein or peptide subunits;
(ii) a length of 100 to 5,000 nm;
(iii) a width of 2 to 20 nm; and
(iv) a length-to-width ratio of 10 or higher; and
(b) a pharmaceutically acceptable carrier,
wherein the filamentous agent is not aggregated in the composition.


SEQUENCE LISTING (4 sequences)

SEQ ID NO: 1 - protein, 4 aa, Artificial Sequence
  synthetic
DGEA

SEQ ID NO: 2 - protein, 5 aa, Artificial Sequence
  synthetic
IRVVM

SEQ ID NO: 3 - protein, 5 aa, Artificial Sequence
  synthetic
PHSRN

SEQ ID NO: 4 - protein, 8 aa, Artificial Sequence
  synthetic
RFYVVMWK